(19) United States
(12) Patent Application Publication
(10) Pub. No.: US 2023/0303706 A1
(43) Pub. Date: Sep. 28, 2023

(54) MONOCYTE

(71) Applicant: University of Surrey, Guildford (GB)

(72) Inventors: Fernando Oneissi Martinez Estrada, Guildford (GB); Theo William Combes, Guildford (GB)

(21) Appl. No.: 17/995,815

(22) PCT Filed: Apr. 9, 2021
(86) PCT No.: PCT/GB2021/050869

§ 371 Date: Oct. 7, 2022

(30) Foreign Application Priority Data
Apr. 9, 2020 (GB) 2005274.2

(51) Int. Cl. C07K 16/28; G01N 33/569

(52) U.S. Cl. CPC C07K 16/2866; G01N 33/56972

(57) ABSTRACT

The invention relates to a novel pan-monocyte/pan-monocyte-derived cell biomarker and the use of an antibody immunospecific to the marker. The invention extends to methods for isolating and detecting human monocytes and monocyte-derived cells from biological samples, such as blood, based on the marker and the antibody.

The present invention relates to monocytes and monocyte-derived cells, and particularly, although not exclusively, to a novel pan-monocyte/pan- monocyte-derived cell biomarker and the use of an antibody immunospecific to the marker. The invention extends to methods for isolating and detecting human monocytes and monocyte-derived cells from biological samples, such as blood, based on the marker and the antibody.

Monocytes make up approximately 10% of white blood cells (i.e. leukocytes) in humans, and can differentiate into macrophages and myeloid lineage dendritic cells. There are at least three sub-classes of monocytes in human blood based on their phenotypic receptors. Firstly, the classical monocyte is characterised by high level expression of the CD₁₄ cell surface receptor (i.e. CD₁₄++ CD₁₆ monocyte). Secondly, the non-classical monocyte shows low level expression of CD₁₄ and additional co-expression of the CD₁₆ receptor (i.e. CD₁₄++ CD₁₆++ monocyte). Thirdly, the intermediate monocyte with high level expression of CD₁₄ and low-level expression of CD₁₆ (i.e. CD₁₄++ CD₁₆⁺ monocyte).

A monocyte count is part of a complete blood count and is expressed either as a percentage of monocytes among white blood cells or as absolute numbers. Both may be useful, but these cells are only valid diagnostic tools when monocyte subsets are determined. Monocytosis is the state of excess monocytes in peripheral blood, and it may be indicative of various disease conditions, such as chronic inflammation, immune-mediated diseases, and atherosclerosis etc. A high count of CD₁₄ CD₁₆⁺⁺ monocytes is found in sepsis, and in the field of atherosclerosis, high numbers of the CD₁₄++ CD₁₆+ intermediate monocytes were shown to be predictive of cardiovascular events in at risk populations.

Currently, in order to isolate pan monocytes from blood, it is necessary to use a negative selection technique using multiple different antibodies binding to CD₃, CD₇, CD₁₆, CD₁₉, CD₅₆, CD₁₂₃, and Glycophorin A.

The CD₁₄ and CD₁₆ markers are in the proximity of lineage determining cytokine receptors in the genome (Goyert et al., 1988; Mahaweni et al., 2018). However, the inventors believe that they are, in fact, downstream markers that fail to fully capture monocyte ontogeny. A large number of studies have used this classification to characterise the transcriptome, proteome and functional properties of these cells (Martinez, 2009). Monocyte subsets have also been studied in a large number of pathologies. To identify subsets, gating strategies focus on the shape of monocytes to exclude neutrophils, expression of CD₁₄ and CD₁₆, and exclusion of cells of the lymphoid lineage. However, these combined gates and exclusion criteria pose major limitations and poor reproducibility in clinical settings.

There is, therefore, a significant need for improved monocyte markers and antibodies which facilitate or improve monocyte isolation and detection from biological samples.

The present invention arises from the inventors’ work in attempting to overcome the problems associated with the prior art.

The inventors investigated the co-expression of lineage determining receptors in human blood. To their surprise, they found that CSF-₁R may be used as a pan-monocyte marker. The inventors were able to isolate all monocytes using a CSF-₁R antibody, which cannot be achieved with the markers CD₁₄ or CD₁₆ alone, and did not require any additional antibodies. The inventors have shown that CSF-₁R may be used as a main monocyte marker that enables accurate isolation, quantification and a novel monocyte view from an ontogeny perspective. Furthermore, the inventors have identified a fourth sub-set of CSF₁R+ cells that do not express CD₁₄ or CD₁₆ and thus would not be detected using current isolation and detection approaches.

Antibodies that bind to CSF-₁R are known. For example, emactuzumab is a humanized monoclonal antibody as described in US20110165156, which binds to CSF-₁R, and has been utilised for the treatment of a number of conditions. However, to date, there has been no recorded use of the antibody for detecting and isolating monocytes. The inventors have surprisingly shown that antibodies binding to CSF-₁R alone may be used to pull down all monocyte populations present in a biological sample, and are therefore able to be used as a pan monocyte isolation antibodies that do not require the use of any other antibodies or isolation steps.

Thus, according to a first aspect of the invention, there is provided the use of an antibody, or antigen-binding fragment thereof, which binds to colony stimulating factor 1 receptor (CSF-₁R), or a variant or fragment thereof, to isolate monocytes and/or monocyte-derived cells from a biological sample.

The monocyte-derived cell may be a macrophage and/or a myeloid lineage dendritic cell. Preferably, the monocyte-derived cell is a macrophage. Preferably, the monocyte-derived cell is a myeloid lineage dendritic cell.

Preferably, the biological sample is a human or murine biological sample. Most preferably, the biological sample is a human biological sample.

The biological sample may be tissue or a biological fluid.

The biological sample may be any material that is obtainable from the subject from which monocytes are obtainable. Furthermore, the sample may be blood, plasma, serum, spinal fluid, urine, sweat, saliva, tears, breast aspirate, breast milk, prostate fluid, seminal fluid, vaginal fluid, stool, cervical scraping, cytes, amniotic fluid, intraocular fluid, mucous, moisture in breath, animal tissue, cell lysates, tumour tissue, hair, skin, buccal scrapings, lymph, interstitial fluid, nails, bone marrow, cartilage, prions, bone powder, ear wax, lymph, granuloma, cancer biopsy or combinations thereof.

The sample may be a liquid aspirate. For example, the sample may be bronchial alveolar lavage (BAL), ascites, pleural lavage, peritoneal lavage or pericardial lavage.

In one embodiment, the biological sample comprises a tissue, for example a cancer biopsy or a granuloma. The tissue may be further processed before the method is performed. For instance, the tissue may be enzymatically digested without degrading CSF-R₁.

In one preferred embodiment, the biological sample comprises a blood sample. The blood may be venous or arterial blood. Blood samples may be assayed immediately. Alternatively, the blood sample may be stored at low temperatures, for example in a fridge or even frozen before the method is conducted. Alternatively, the blood sample may be stored at room temperature, for example between 18 to 22° C., before the method is conducted. The blood sample may comprise comprises blood serum. The blood sample may comprise blood plasma. Preferably, however the detection is carried out on whole blood and most preferably the blood sample is peripheral blood. The method may be performed with at least 1 ml of blood, at least 500 µl of blood, at least 400 µl of blood, at least 400 µl of blood, at least 300 µl of blood, at least 200 µl of blood, at least 100 µl of blood, or at least 50 µl of blood.

The blood may be further processed before the use of the first aspect is performed. For instance, an anticoagulant, such as citrate (such as sodium citrate), hirudin, heparin, PPACK, or sodium fluoride may be added. Thus, the sample collection container may contain an anticoagulant in order to prevent the blood sample from clotting.

In one embodiment, when the cell is a monocyte, preferably the sample is a blood sample. In one embodiment, when the cell is a monocyte-derived cell, preferably the sample is a tissue sample.

The skilled person would understand that colony stimulating factor 1 receptor (CSF-R₁) may also be referred to as Cluster of Differentiation 115 (CD₁₁₅) or macrophage colony-stimulating factor receptor (M-CSFR).

In one embodiment, CSF-R₁ is provided by GeneBank ID 1436, which is provided herein as SEQ ID No: 1, as follows:

MGPGVLLLLLVATAWHGQGIPVIEPSVPELVVKPGATVTLRCVGNGSVEW
DGPPSPHWTLYSDGSSSILSTNNATFQNTGTYRCTEPGDPLGGSAAIHLY
VKDPARPWNVLAQEVVVFEDQDALLPCLLTDPVLEAGVSLVRVRGRPLMR
HTNYSFSPWHGFTIHRAKFIQSQDYQCSALMGGRKVMSISIRLKVQKVIP
GPPALTLVPAELVRIRGEAAQIVCSASSVDVNFDVFLQHNNTKLAIPQQS
DFHNNRYQKVLTLNLDQVDFQHAGNYSCVASNVQGKHSTSMFFRVVESAY
LNLSSEQNLIQEVTVGEGLNLKVMVEAYPGLQGFNWTYLGPFSDHQPEPK
LANATTKDTYRHTFTLSLPRLKPSEAGRYSFLARNPGGWRALTFELTLRY
PPEVSVIWTFINGSGTLLCAASGYPQPNVTWLQCSGHTDRCDEAQVLQVW
DDPYPEVLSQEPFHKVTVQSLLTVETLEHNQTYECRAHNSVGSGSWAFIP
ISAGAHTHPPDEFLFTPVVVACMSIMALLLLLLLLLLYKYKQKPKYQVRW
KIIESYEGNSYTFIDPTQLPYNEKWEFPRNNLQFGKTLGAGAFGKVVEAT
AFGLGKEDAVLKVAVKMLKSTAHADEKEALMSELKIMSHLGQHENIVNLL
GACTHGGPVLVITEYCCYGDLLNFLRRKAEAMLGPSLSPGQDPEGGVDYK
NIHLEKKYVRRDSGFSSQGVDTYVEMRPVSTSSNDSFSEQDLDKEDGRPL
ELRDLLHFSSQVAQGMAFLASKNCIHRDVAARNVLLTNGHVAKIGDFGLA
RDIMNDSNYIVKGNARLPVKWMAPESIFDCVYTVQSDVWSYGILLWEIFS
LGLNPYPGILVNSKFYKLVKDGYQMAQPAFAPKNIYSIMQACWALEPTHR
PTFQQICSFLQEQAQEDRRERDYTNLPSSSRSGGSGSSSSELEEESSSEH
LTCCEQGDIAQPLLQPNNYQFC
[SEQ ID No: 1]

Accordingly, CSF-R₁ preferably comprises or consists of an amino acid sequence as substantially as set out in SEQ ID No: 1, or a variant or fragment thereof.

The monocyte may be CD₁₄ positive and/or CD₁₆ positive.

Preferably, the antibody is capable of isolating all sub-classes of monocytes.

The inventors were especially surprised to observe that CD₁₄ negative and CD₁₆ negative cells, which are monocyte-derived myeloid lineage dendritic cells could be isolated or detected using the antibody. Hence, preferably the antibody, or antigen binding fragment thereof, may be used to isolate a CD₁₄ negative and CD₁₆ negative monocyte-derived myeloid dendritic cells.

The CD₁₄ negative and CD₁₆ negative monocyte derived myeloid lineage dendritic cell may be defined by the expression of CD₁C.

CD₁C may also be referred to as Cortical Thymocyte Antigen.

Thus, preferably, the antibody, or antigen binding fragment thereof, may be used to isolate:
i) a CD₁₄ positive and CD₁₆ positive monocyte;
ii) a CD₁₄ negative and CD₁₆ positive monocyte;
iii) a CD₁₄ positive and CD₁₆ negative monocyte; and/or
iv) a CD₁₄ negative and CD₁₆ negative monocyte-derived myeloid lineage dendritic cell.

Preferably the antibody, or antigen binding fragment thereof, may be used to isolate:
i) a CD₁₄ positive and CD₁₆ positive monocyte;
ii) a CD₁₄ negative and CD₁₆ positive monocyte;
iii) a CD₁₄ positive and CD₁₆ negative monocyte; and
iv) a CD₁₄ negative and CD₁₆ negative monocyte-derived myeloid lineage dendritic cell.

Thus, preferably, the antibody, or antigen binding fragment thereof, may be used to isolate:
i) a CD₁₄ positive and CD₁₆ positive monocyte;
ii) a CD₁₄ negative and CD₁₆ positive monocyte;
iii) a CD₁₄ positive and CD₁₆ negative monocyte; and/or
iv) a CD₁₄ negative, CD₁₆ negative and CD₁C monocyte-derived myeloid lineage dendritic cell.

Thus, preferably, the antibody, or antigen binding fragment thereof, may be used to isolate:
i) a CD₁₄ positive and CD₁₆ positive monocyte;
ii) a CD₁₄ negative and CD₁₆ positive monocyte;
iii) a CD₁₄ positive and CD₁₆ negative monocyte; and
iv) a CD₁₄ negative, CD₁₆ negative and CD₁C positive monocyte-derived lineage myeloid dendritic cell.

Preferably, the use does not comprise the use of any other antibody, or antigen binding fragment thereof.

The use of the first aspect may be performed by florescence-activated cell sorting (FACS), magnetic activated cell separation or buoyancy activated cell separation. Such methods would be known to those skilled in the art.

Thus, the use may comprise use of the antibody or antigen binding fragment thereof, conjugated to a fluorophore, a magnetic particle or to a glass microbubble.

Preferably, the use may comprise use of the antibody or antigen binding fragment thereof conjugated to a magnetic particle. Thus, the use may preferably comprise magnetic activated cell separation, such as immunomagnetic cell separation or affinity magnetic cell separation. Such methods would be known to those skilled in the art.

The skilled person would understand that immunomagnetic separation is a laboratory tool that can efficiently isolate cells out of body fluid or cultured cells. DNA analysis have supported the combined use of both this technique and Polymerase Chain Reaction (PCR), thus the technical may be used in combination with PCR. Suitable immunomagnetic separation systems include the Dynal system (Dynal [UK] Ltd., Wirral, Mersyside, UK; Dynal, Inc., Lake Success, NY) and the MACS system, produced by Miltenyi Biotech (Miltenyi Biotech Ltd., Bisley, Surrey, UK; Miltenyi Biotech Inc., Auburn, CA).

The skilled person would understand that immunomagnetic separation may be used to isolate of cells, proteins, and nucleic acids within a cell culture or body fluid through the specific capture of biomolecules through the attachment of small-magnetized particles, beads, containing antibodies and lectins. These beads may be coated to bind to targeted biomolecules, gently separated and goes through multiple cycles of washing to obtain targeted molecules bound to these super paramagnetic beads, which can differentiate based on strength of magnetic field and targeted molecules. These may then be eluted to collect supernatant and then are able to determine the concentration of specifically targeted biomolecules.

A mixture of cell populations may be put into a magnetic field where cells, which are then are attached to super paramagnetic beads, in one example this may Dynabeads (4.5-µm), which will remain once excess substrate is removed binding to targeted antigen. Dynabeads may consist of iron-containing cores, which is covered by a thin layer of a polymer shell allowing the absorption of biomolecules. The beads may be coated with the antibody or antigen binding fragment according to the first aspect; the linkage between magnetized beads coated materials may be a cleavable DNA linker, to enable cell separation from the beads when the culturing of cells is more desirable.

Many other commercially available beads have the same principles of separation; however, the presence and different strengths of magnetic fields may require certain sizes of beads, based on the ramifications of the separation of the cell population, which would be known to those skilled in the art.

Preferably, the use comprises use of FACS, wherein the use comprises:
i) contacting a biological sample that comprises a monocyte and/or a monocyte-derived cell with a fluorescently labelled antibody, or antigen-binding fragment thereof, of the first aspect; and
ii) sorting cells present in the biological sample based on their fluorescence; and
iii) collecting a monocyte and/or a monocyte-derived cell present in the biological sample that are bound to the fluorescently labelled antibody, thereby isolating a monocyte and/or a monocyte-derived cell that is present in the biological sample.

The skilled person would appreciate that cells may be sorted based on fluorescence emission profile and/or fluorescence intensity, which will depend on the fluorescent label used and which are well known in the art.

Preferably, the use comprises use of magnetic cell sorting, wherein the use comprises:
i) contacting a biological sample that comprises a monocyte and/or a monocyte-derived cell with magnetic beads coated with an antibody or antigen-binding fragment thereof of the first aspect; and
ii) placing the sample resulting from step i) in a magnetic field wherein the monocyte and/or a monocyte-derived cells bound to the magnetic beads are separated from other cells present in the sample;
iii) washing the sample to remove other cells from the sample; and
iii) collecting the monocyte and/or monocyte-derived cells present in the biological sample that are bound to the magnetic beads, thereby isolating a monocyte and/or a monocyte-derived cell that is present in the biological sample.

The invention extends to both whole antibodies (i.e. immunoglobulins) with immunospecificity for CSF-₁R, as well as to antigen-binding fragments or regions of the corresponding full-length antibody which bind to CSF-₁R.

In a preferred embodiment, the antibody is an antibody derived or derivable from hybridoma clone number ₉-₄D₂-₁E₄, or the antibody is an antibody derived or derivable from hybridoma clone number ₁₂-₃A₃-₁B₁₀.

In a preferred embodiment, the human antibody is emactuzumab, which may also be referred to as RG₇₁₅₅ and may be the CD₁₁₅ binding antibody as defined in US20110165156.

Preferably, the antibody comprises a heavy chain variable domain of SEQ ID No: 2, which is provided herein as follows:

QVQLVQSGAEVKKPGASVKVSCKASGYTFTSYDISWVRQAPGQGLEWMGV
IWTDGGTNYAQKLQGRVTMTTDTSTSTAYMELRSLRSDDTAVYYCARDQR
LYFDVWGQGTTV
[SEQ ID NO: 2]

Accordingly, preferably the antibody or antigen binding fragment thereof comprises or consists of a heavy chain variable domain comprising the amino acid sequence as substantially set out in SEQ ID No: 2, or a variant or fragment thereof.

Preferably, the antibody, or antigen binding fragment thereof, comprises a heavy chain variable domain that comprises a CDR₃ region of SEQ ID No ₃, provided herein as follows:

DQRLYFDV [SEQ ID No: 3]

Accordingly, preferably the antibody or antigen binding fragment thereof comprises or consists of a heavy chain variable domain comprising a CDR₃ region comprising the amino acid sequence as substantially set out in SEQ ID No: ₃ or a variant or fragment thereof.

Preferably, the antibody, or antigen binding fragment thereof, comprises a heavy chain variable domain that comprises a CDR₂ region of SEQ ID No 4, provided herein as follows:

VIWTDGGTNYAQKLQG [SEQ ID
No: 4]

Accordingly, preferably the antibody or antigen binding fragment thereof comprises or consists of a heavy chain variable domain comprising a CDR₃ region comprising or consisting of the amino acid sequence as substantially set out in SEQ ID No: ₄ or a variant or fragment thereof.

Preferably, the antibody, or antigen binding fragment thereof, comprises a heavy chain variable domain that comprises a CDR₁ region of SEQ ID No ₅, provided herein as follows:

SYDIS [SEQ ID No: 5]

Accordingly, preferably the antibody or antigen binding fragment thereof comprises a heavy chain variable domain comprising a CDR₁ region comprising or consisting of the amino acid sequence as substantially set out in SEQ ID No: ₅ or a variant or fragment thereof.

Preferably, the antibody comprises a light chain variable domain VL of SEQ ID No 6, which is provided herein as follows:

DIQMTQSPSSLSASVGDRVTITCRASEDVNTYVSWYQQKPGKAPKLLIYA
ASNRYTGVPSRFSGSGSGTDFTLTISSLQPEDFATYYCQQSFSYPTFGQG
TKLEIK
[SEQ ID NO: 6]

Accordingly, preferably the light chain variable domain of the antibody or antigen binding fragment thereof comprises or consists of the amino acid sequence as substantially set out in SEQ ID No: 6, or a variant or fragment thereof.

Preferably, the antibody, or antigen binding fragment thereof, comprises a light chain variable domain that comprises a CDR₃ region of SEQ ID No ₇, provided herein as follows:

QQSFSYPT [SEQ ID No: 7]

Accordingly, preferably the antibody or antigen binding fragment thereof comprises a light chain variable domain comprising a CDR₃ region comprising or consisting of the amino acid sequence as substantially set out in set out in SEQ ID No: ₇, or a variant or fragment thereof.

Preferably, the antibody, or antigen binding fragment thereof, comprises a light chain variable domain that comprises a CDR₂ region of SEQ ID No 8, provided herein as follows:

AASNRYT [SEQ ID No: 8]

Accordingly, preferably the antibody or antigen binding fragment thereof comprises a light chain variable domain comprising a CDR₂ region comprising or consisting of the amino acid sequence as substantially set out in set out in SEQ ID No: 8, or a variant or fragment thereof.

Preferably, the antibody, or antigen binding fragment thereof, comprises a light chain variable domain that comprises a CDR₁ region of SEQ ID No 9, provided herein as follows:

RASEDVNTYVS [SEQ ID No: 9
]

Accordingly, preferably the antibody or antigen binding fragment thereof comprises a light chain variable domain comprising a CDR₁ region comprising or consisting of the amino acid sequence as substantially set out in set out in SEQ ID No: 9, or a variant or fragment thereof.

Therefore, in one preferred embodiment, the antibody, or antigen binding fragment thereof comprises a heavy chain of SEQ ID No: 10, which is provided herein as follows:

QVQLVQSGAEVKKPGASVKVSCKASGYTFTSYDISWVRQAPGQGLEWMGV
IWTDGGTNYAQKLQGRVTMTTDTSTSTAYMELRSLRSDDTAVYYCARDQR
LYFDVWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
[SEQ ID NO: 10]

Accordingly, preferably, the antibody, or antigen binding fragment thereof, comprises a heavy chain region comprising or consisting of the amino acid sequence as substantially set out in set out in SEQ ID No: 10, or a variant or fragment thereof.

Therefore, in one preferred embodiment, the antibody, or antigen binding fragment thereof, comprises a light chain of SEQ ID No: 11, which is provided herein as follows:

DIQMTQSPSSLSASVGDRVTITCRASEDVNTYVSWYQQKPGKAPKLLIYA
ASNRYTGVPSRFSGSGSGTDFTLTISSLQPEDFATYYCQQSFSYPTFGQG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
[SEQ ID No: 11]

Accordingly, preferably, the antibody, or antigen binding fragment thereof, comprises a heavy chain region comprising or consisting of the amino acid sequence as substantially set out in set out in SEQ ID No: 11, or a variant or fragment thereof.

The invention extends to both whole antibodies (i.e. immunoglobulins) with immunospecificity for CSF-₁R, as well as to antigen-binding fragments or regions of the corresponding full-length antibody.

The antibody or antigen-binding fragment thereof may be monovalent, divalent or polyvalent.

Monovalent antibodies are dimers (HL) comprising a heavy (H) chain associated by a disulphide bridge with a light chain (L). Divalent antibodies are tetramer (H₂L₂) comprising two dimers associated by at least one disulphide bridge. Polyvalent antibodies may also be produced, for example by linking multiple dimers. The basic structure of an antibody molecule consists of two identical light chains and two identical heavy chains which associate non-covalently and can be linked by disulphide bonds. Each heavy and light chain contains an amino-terminal variable region of about 110 amino acids, and constant sequences in the remainder of the chain. The variable region includes several hypervariable regions, or Complementarity Determining Regions (CDRs), that form the antigen-binding site of the antibody molecule and determine its specificity for the antigen, i.e. CSF-₁R, or variant or fragment thereof (e.g. an epitope). On either side of the CDRs of the heavy and light chains is a framework region, a relatively conserved sequence of amino acids that anchors and orients the CDRs. Antibody fragments may include a bi-specific antibody (BsAb) or a chimeric antigen receptor (CAR).

The constant region consists of one of five heavy chain sequences (µ, γ, ζ, α, or ε) and one of two light chain sequences (κ or λ). The heavy chain constant region sequences determine the isotype of the antibody and the effector functions of the molecule.

Preferably, the antibody or antigen-binding fragment thereof is isolated or purified.

In one preferred embodiment, the antibody or antigen-binding fragment thereof comprises a polyclonal antibody, or an antigen-binding fragment thereof. The antibody or antigen-binding fragment thereof may be generated in a rabbit, mouse or rat.

In another preferred embodiment, the antibody or antigen-binding fragment thereof comprises a monoclonal antibody or an antigen-binding fragment thereof. Preferably, the antibody of the invention is a human antibody.

As used herein, the term “human antibody” can mean an antibody, such as a monoclonal antibody, which comprises substantially the same heavy and light chain CDR amino acid sequences as found in a particular human antibody exhibiting immunospecificity for CSF-R₁, or a variant or fragment thereof. An amino acid sequence, which is substantially the same as a heavy or light chain CDR, exhibits a considerable amount of sequence identity when compared to a reference sequence. Such identity is definitively known or recognizable as representing the amino acid sequence of the particular human antibody. Substantially the same heavy and light chain CDR amino acid sequence can have, for example, minor modifications or conservative substitutions of amino acids. Such a human antibody maintains its function of selectively binding to CSF-R₁ or a variant or fragment thereof.

The term “human monoclonal antibody” can include a monoclonal antibody with substantially or entirely human CDR amino acid sequences produced, for example by recombinant methods such as production by a phage library, by lymphocytes or by hybridoma cells.

The term “humanised antibody” can mean an antibody from a non-human species (e.g. mouse or rabbit) whose protein sequences have been modified to increase their similarity to antibodies produced naturally in humans.

The antibody may be a recombinant antibody. The term “recombinant human antibody” can include a human antibody produced using recombinant DNA technology.

The term “antigen-binding region” can mean a region of the antibody having specific binding affinity for its target antigen, for example, CSF-R₁, or a variant or fragment thereof. Preferably, the fragment is an epitope. The binding region may be a hypervariable CDR or a functional portion thereof. The term “functional portion” of a CDR can mean a sequence within the CDR which shows specific affinity for the target antigen. The functional portion of a CDR may comprise a ligand which specifically binds to CSF-R₁ or a fragment thereof.

The term “CDR” can mean a hypervariable region in the heavy and light variable chains. There may be one, two, three or more CDRs in each of the heavy and light chains of the antibody. Normally, there are at least three CDRs on each chain which, when configured together, form the antigen-binding site, i.e. the three-dimensional combining site with which the antigen binds or specifically reacts. It has however been postulated that there may be four CDRs in the heavy chains of some antibodies.

The definition of CDR also includes overlapping or subsets of amino acid residues when compared against each other. The exact residue numbers which encompass a particular CDR or a functional portion thereof will vary depending on the sequence and size of the CDR. Those skilled in the art can routinely determine which residues comprise a particular CDR given the variable region amino acid sequence of the antibody.

The term “functional fragment” of an antibody can mean a portion of the antibody which retains a functional activity. A functional activity can be, for example antigen binding activity or specificity. A functional activity can also be, for example, an effector function provided by an antibody constant region. The term “functional fragment” is also intended to include, for example, fragments produced by protease digestion or reduction of a human monoclonal antibody and by recombinant DNA methods known to those skilled in the art. Human monoclonal antibody functional fragments include, for example individual heavy or light chains and fragments thereof, such as VL, VH and Fd; monovalent fragments, such as Fv, Fab, and Fab′; bivalent fragments such as F(ab′)₂; single chain Fv (scFv); and Fc fragments.

The term “VL fragment” can mean a fragment of the light chain of a human monoclonal antibody which includes all or part of the light chain variable region, including the CDRs. A VL fragment can further include light chain constant region sequences.

The term “VH fragment” can means a fragment of the heavy chain of a human monoclonal antibody which includes all or part of the heavy chain variable region, including the CDRs.

The term “Fd fragment” can mean the heavy chain variable region coupled to the first heavy chain constant region, i.e. VH and CH-₁. The “Fd fragment” does not include the light chain, or the second and third constant regions of the heavy chain.

The term “Fv fragment” can mean a monovalent antigen-binding fragment of a human monoclonal antibody, including all or part of the variable regions of the heavy and light chains, and absent of the constant regions of the heavy and light chains. The variable regions of the heavy and light chains include, for example, the CDRs. For example, an Fv fragment includes all or part of the amino terminal variable region of about 110 amino acids of both the heavy and light chains.

The term “Fab fragment” can mean a monovalent antigen-binding fragment of a human monoclonal antibody that is larger than an Fv fragment. For example, a Fab fragment includes the variable regions, and all or part of the first constant domain of the heavy and light chains. Thus, a Fab fragment additionally includes, for example, amino acid residues from about 110 to about 220 of the heavy and light chains.

The term “Fab′ fragment” can mean a monovalent antigen-binding fragment of a human monoclonal antibody that is larger than a Fab fragment. For example, a Fab′ fragment includes all of the light chain, all of the variable region of the heavy chain, and all or part of the first and second constant domains of the heavy chain. For example, a Fab′ fragment can additionally include some or all of amino acid residues 220 to 330 of the heavy chain.

The term “F(ab′)₂ fragment” can mean a bivalent antigen-binding fragment of a human monoclonal antibody. An F(ab′)₂ fragment includes, for example, all or part of the variable regions of two heavy chains-and two light chains, and can further include all or part of the first constant domains of two heavy chains and two light chains.

The term “single chain Fv (scFv)” can mean a fusion of the variable regions of the heavy (VH) and light chains (VL) connected with a short linker peptide.

The term “bispecific antibody (BsAb)” can mean a bispecific antibody comprising two scFv linked to each other by a shorter linked peptide.

One skilled in the art knows that the exact boundaries of a fragment of an antibody are not important, so long as the fragment maintains a functional activity. Using well-known recombinant methods, one skilled in the art can engineer a polynucleotide sequence to express a functional fragment with any endpoints desired for a particular application. A functional fragment of the antibody may comprise or consist of a fragment with substantially the same heavy and light chain variable regions as the human antibody.

Preferably, the antigen-binding fragment thereof, with respect to the first aspect of the invention, is CSF-R₁ -specific or immunospecific for an epitope within CSF-R₁. The antigen-binding fragment thereof may comprise or consist of any of the fragments selected from a group consisting of VH, VL, Fd, Fv, Fab, Fab′, scFv, F (ab′)₂ and Fc fragment.

The antigen-binding fragment thereof may comprise or consist of any one of the antigen binding region sequences of the VL, any one of the antigen binding region sequences of the VH, or a combination of VL and VH antigen binding regions of a human antibody. The appropriate number and combination of VH and VL antigen binding region sequences may be determined by those skilled in the art depending on the desired affinity and specificity and the intended use of the antigen-binding fragment. Functional fragments or antigen-binding fragments of antibodies may be readily produced and isolated using methods well known to those skilled in the art. Such methods include, for example, proteolytic methods, recombinant methods and chemical synthesis. Proteolytic methods for the isolation of functional fragments comprise using human antibodies as a starting material. Enzymes suitable for proteolysis of human immunoglobulins may include, for example, papain, and pepsin. The appropriate enzyme may be readily chosen by one skilled in the art, depending on, for example, whether monovalent or bivalent fragments are required. For example, papain cleavage results in two monovalent Fab′ fragments that bind antigen and an Fc fragment. Pepsin cleavage, for example, results in a bivalent F (ab′) fragment. An F (ab′)₂ fragment of the invention may be further reduced using, for example, DTT or 2-mercaptoethanol to produce two monovalent Fab′ fragments.

Functional or antigen-binding fragments of antibodies produced by proteolysis may be purified by affinity and column chromatographic procedures. For example, undigested antibodies and Fc fragments may be removed by binding to protein A. Additionally, functional fragments may be purified by virtue of their charge and size, using, for example, ion exchange and gel filtration chromatography. Such methods are well known to those skilled in the art.

The antibody or antigen-binding fragment thereof may be produced by recombinant methodology. Preferably, one initially isolates a polynucleotide encoding desired regions of the antibody heavy and light chains. Such regions may include, for example, all or part of the variable region of the heavy and light chains. Preferably, such regions can particularly include the antigen binding regions of the heavy and light chains, preferably the antigen binding sites, most preferably the CDRs.

The polynucleotide encoding the antibody or antigen-binding fragment thereof according to the invention may be produced using methods known to those skilled in the art. The polynucleotide encoding the antibody or antigen-binding fragment thereof may be directly synthesized by methods of oligonucleotide synthesis known in the art. Alternatively, smaller fragments may be synthesized and joined to form a larger functional fragment using recombinant methods known in the art.

As used herein, the term “immunospecificity” can mean the binding region is capable of immunoreacting with CSF-R₁, or a variant or fragment thereof, by specifically binding therewith. The antibody or antigen-binding fragment thereof can selectively interact with an antigen (e.g. CSF-R₁ or a variant or fragment thereof) with an affinity constant of approximately 10⁻⁵ to 10⁻¹³ M⁻¹, preferably 10⁻⁶ to 10⁻⁹ M⁻¹, even more preferably, 10⁻¹⁰ to 10⁻¹² M⁻¹.

The term “immunoreact” can mean the binding region is capable of eliciting an immune response upon binding CSF-R₁, or an epitope thereof.

The term “epitope” can mean any region of an antigen with the ability to elicit, and combine with, a binding region of the antibody or antigen-binding fragment thereof.

Advantageously, the inventors have been able to isolate all monocyte types and monocyte-derived cells that are present in a sample using the CSF-₁R antibody of the first aspect (i.e. a CD₁₄ positive, CD₁₆ positive monocyte; a CD₁₄ negative, CD₁₆ positive monocyte; a CD₁₄ positive, CD₁₆ negative monocyte; and a CD₁₄ negative, CD₁₆ negative monocyte-derived myeloid lineage dendritic cell), which is not possible using the markers CD₁₄ and/or CD₁₆ alone, and which does not require the use of any additional antibodies or isolation steps.

Thus, according to a second aspect of the invention, there is provided a method of isolating a monocyte and/or a monocyte derived cell from a biological sample, the method comprising:
i) contacting a biological sample comprising a monocyte and/or a monocyte-derived cell with the antibody, or antigen-binding fragment thereof, according to the first aspect; and
ii) collecting a monocyte and/or monocyte-derived cell present in the biological sample that binds to the antibody, thereby isolating the monocyte and/or monocyte-derived cell.

The monocyte, monocyte-derived cell, biological sample and isolation means may be as defined in the first aspect.

Hence, preferably the method may be used to isolate a CD₁₄ negative and CD₁₆ negative monocyte-derived myeloid lineage dendritic cells.

Preferably, the method may be used to isolate:
i) a CD₁₄ positive and CD₁₆ positive monocyte;
ii) a CD₁₄ negative and CD₁₆ positive monocyte;
iii) a CD₁₄ positive and CD₁₆ negative monocyte; and/or
iv) a CD₁₄ negative and CD₁₆ negative monocyte-derived myeloid lineage dendritic cells.

Preferably, the method may be used to isolate:
i) a CD₁₄ positive and CD₁₆ positive monocyte;
ii) a CD₁₄ negative and CD₁₆ positive monocyte;
iii) a CD₁₄ positive and CD₁₆ negative monocyte; and
iv) a CD₁₄ negative and CD₁₆ negative monocyte-derived myeloid dendritic cells.

Preferably, the method may be used to isolate:
i) a CD₁₄ positive and CD₁₆ positive monocyte;
ii) a CD₁₄ negative and CD₁₆ positive monocyte;
iii) a CD₁₄ positive and CD₁₆ negative monocyte; and/or
iv) a CD₁₄ negative, CD₁₆ negative and CD₁C positive monocyte-derived myeloid lineage dendritic cell.

Preferably, the method may be used to isolate
i) a CD₁₄ positive and CD₁₆ positive monocyte;
ii) a CD₁₄ negative and CD₁₆ positive monocyte;
iii) a CD₁₄ positive and CD₁₆ negative monocyte; and
iv) a CD₁₄ negative, CD₁₆ negative and CD₁C positive monocyte-derived myeloid lineage dendritic cell.

The method of the second aspect may be performed by florescence-activated cell sorting (FACS), magnetic activated cell separation or buoyancy activated cell separation. Such methods would be known to those skilled in the art and may be as defined in the first aspect

Preferably, the method comprises use of FACS, wherein the method comprises:
i) contacting a biological sample that comprises a monocyte and/or a monocyte derived cell with a fluorescently labelled antibody, or antigen-binding fragment thereof, of the first aspect; and
ii) sorting cells present in the biological sample based on their fluorescence; and
iii) collecting a monocyte and/or a monocyte derived cell present in the biological sample that are bound to the fluorescently labelled antibody, thereby isolating a monocyte and/or a monocyte derived cell that is present in the biological sample.

The skilled person would appreciate that cells may be sorted based on fluorescence emission profile and/or fluorescence intensity, which will depend on the fluorescent label used and which are well known in the art.

Preferably, the method comprises use of magnetic cell sorting, wherein the method comprises:
i) contacting a biological sample that comprises a monocyte and/or a monocyte derived cell with magnetic beads coated with an antibody or antigen-binding fragment thereof of the first aspect; and
ii) placing a composition resulting from step i) in a magnetic field wherein the monocyte and/or a monocyte derived cells bound to the magnetic beads are separated from other cells present in the sample;
iii) removing the other cells from the sample; and
iii) collecting the monocyte and/or monocyte derived cells present in the biological sample that are bound to the magnetic beads, thereby isolating a monocyte and/or a monocyte derived cell that is present in the biological sample.

In one embodiment, when the cell is a monocyte, preferably the sample is a blood sample. In one embodiment, when the cell is a monocyte-derived cell, preferably the sample is a tissue sample.

In addition to isolating monocytes and/or monocyte-derived cell, the invention also extends to a method of detecting monocytes and/or monocyte-derived cell.

According to a third aspect of the invention, there is provided a method of detecting a monocyte and/or a monocyte-derived cell present in a biological sample, the method comprising:
i) contacting a biological sample comprising a monocyte with the antibody, or antigen-binding fragment thereof, of the first aspect; and
ii) detecting a monocyte and/or a monocyte-derived cell present in the biological sample by a detection means.

The monocyte, monocyte-derived cell and biological sample may be as defined in the first aspect. The detection means may comprise FACS, as defined in the first and second aspect.

In one embodiment, when the cell is a monocyte, preferably the sample is a blood sample. In one embodiment, when the cell is a monocyte-derived cell, preferably the sample is a tissue sample.

Alternatively, the detection means may comprise immunostaining. Preferably, the detection means comprises immunohistochemistry or immunocytochemistry.

Such detection techniques are well known to those skilled in the art.

Detecting may comprise determining the number of monocytes present in the biological sample. Detecting may comprise determining the number of monocyte-derived cells present in the biological sample.

The method may be used to detect a CD₁₄ negative and CD₁₆ negative monocyte-derived cell.

The method may be used to detect:
i) a CD₁₄ positive and CD₁₆ positive monocyte;
ii) a CD₁₄ negative and CD₁₆ positive monocyte;
iii) a CD₁₄ positive and CD₁₆ negative monocyte; and/or
iv) a CD₁₄ negative and CD₁₆ negative monocyte-derived myeloid dendritic cell.

Preferably, the method may be used to detect:
i) a CD₁₄ positive and CD₁₆ positive monocyte;
ii) a CD₁₄ negative and CD₁₆ positive monocyte;
iii) a CD₁₄ positive and CD₁₆ negative monocyte; and
iv) a CD₁₄ negative and CD₁₆ negative monocyte-derived myeloid lineage dendritic cell.

Preferably, the method may be used to isolate:
i) a CD₁₄ positive and CD₁₆ positive monocyte;
ii) a CD₁₄ negative and CD₁₆ positive monocyte;
iii) a CD₁₄ positive and CD₁₆ negative monocyte; and/or
iv) a CD₁₄ negative, CD₁₆ negative and CD₁C positive monocyte-derived myeloid lineage dendritic cell.

Preferably, the method may be used to isolate
i) a CD₁₄ positive and CD₁₆ positive monocyte;
ii) a CD₁₄ negative and CD₁₆ positive monocyte;
iii) a CD₁₄ positive and CD₁₆ negative monocyte; and
iv) a CD₁₄ negative, CD₁₆ negative and CD₁C positive monocyte-derived myeloid lineage dendritic cell.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1 to 11 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:- (i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty = 15.0, Gap Extension Penalty = 6.66, and Matrix = Identity. For protein alignments: Gap Open Penalty = 10.0, Gap Extension Penalty = 0.2, and Matrix = Gonnet. For DNA and Protein alignments: ENDGAP = -1, and GAPDIST = 4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:- Sequence Identity = (N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3x sodium chloride/sodium citrate (SSC) at approximately 45° C. followed by at least one wash in 0.2x SSC/0.1% SDS at approximately 20-65° C. Alternatively, a substantially similar polypeptide may differ by at least 1, but less than 5, 10, 20, 50 or 100 amino acids from the sequences shown in, for example, SEQ ID Nos: 1 to 11.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:-

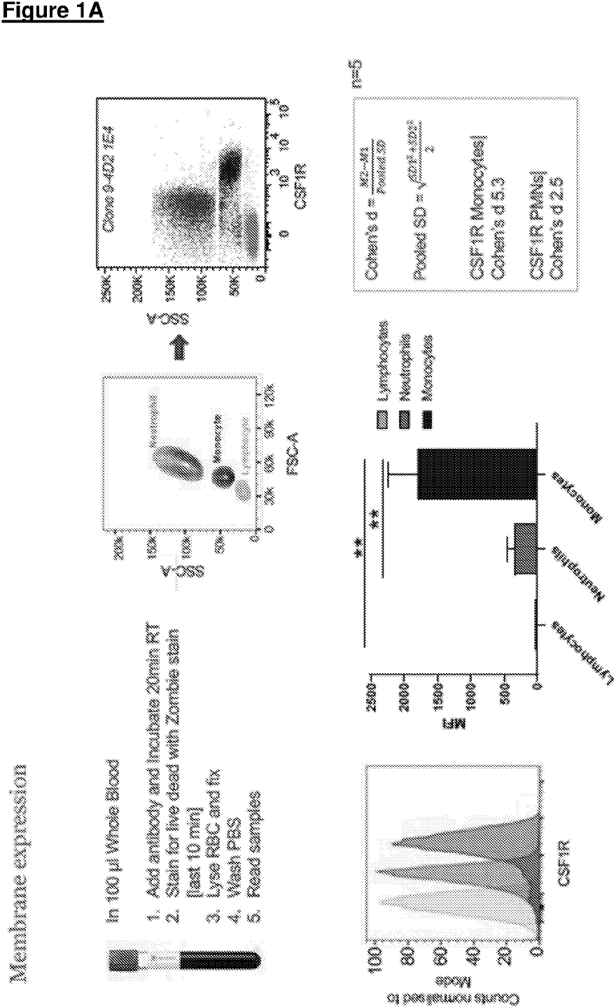

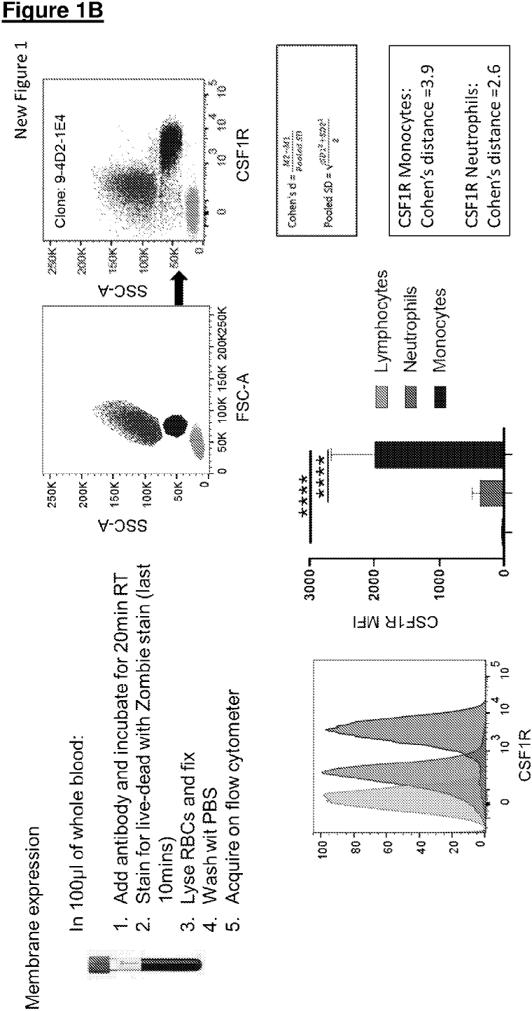

FIGS. 1A and 1B show that CSF-R₁ is a selective monocyte marker in human blood. Monocytes express significantly more CSF₁R than myeloid neutrophils or lymphoid lymphocytes. Monocytes were stained for CSF₁R in whole blood in a rapid staining protocol. Data plotted is the mean with SD for 5 biological replicates.

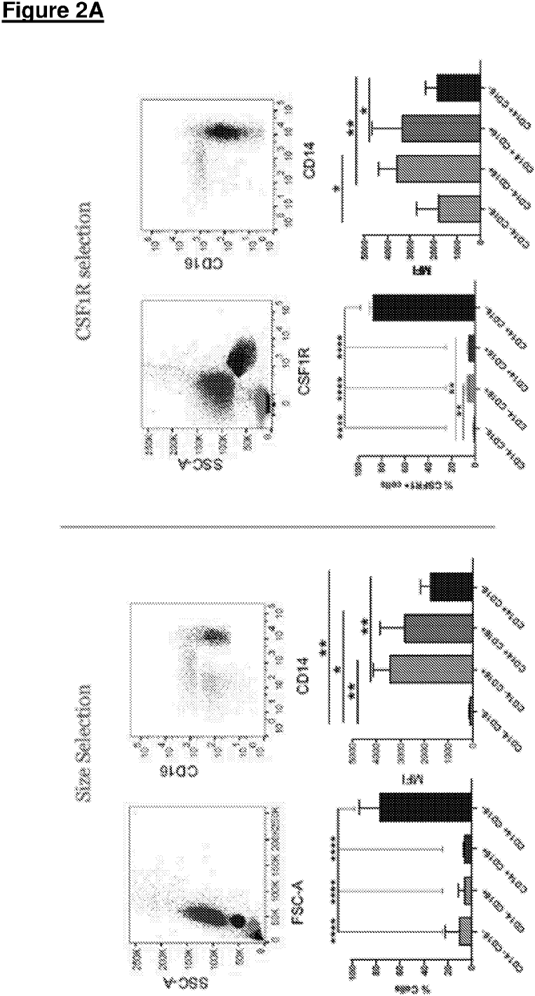

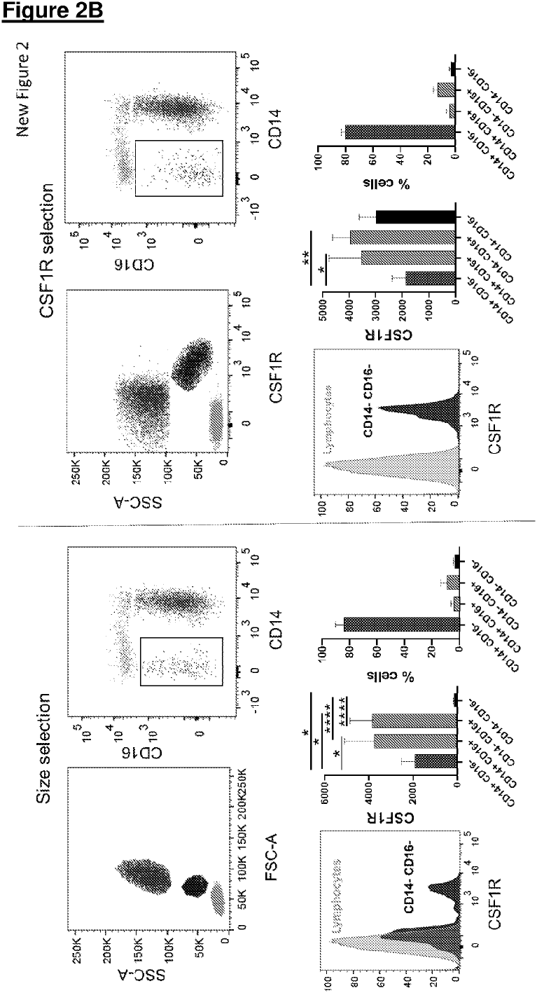

FIGS. 2A and 2B show that CSF-R₁ is highly expressed in all monocyte subsets. Monocytes were stained for flow cytometry in whole blood. Gating monocytes based on size and granularity (left side) and then CD₁₄ vs CD₁₆ selects for the 3 traditional monocyte subsets plus a large double negative group that has a low median fluorescent intensity for CSF-R₁, suggests that this population is mainly contaminating lymphocytes. However, on the right side, selecting monocytes based on CSF-R₁ positivity, generates a much cleaner CD₁₄ vs CD₁₆ pairwise plot, with a smaller double negative population (orange), that has a median fluorescent intensity for CSF-R₁ that is comparable to the other monocyte subsets. Data plotted is the mean with SD for 5 biological replicates.

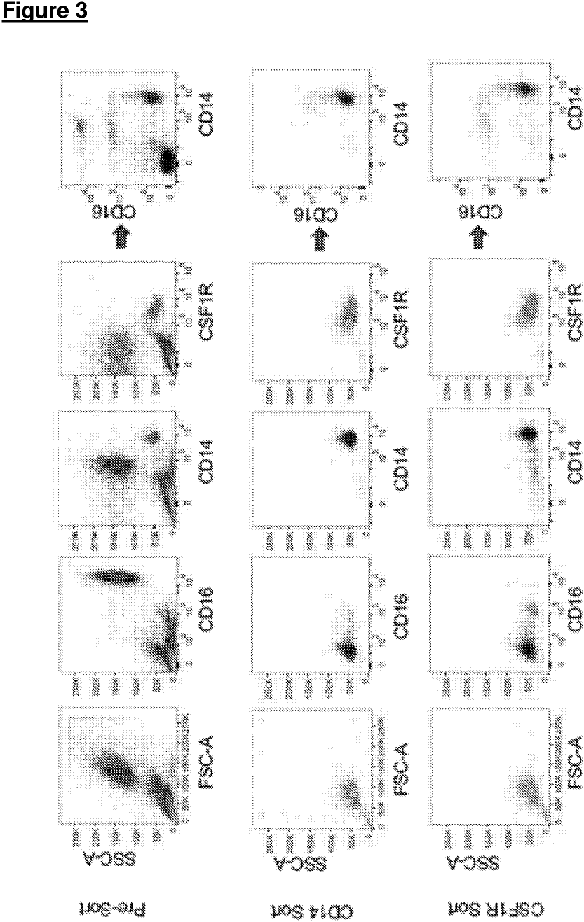

FIG. 3 shows that CSF-R₁ is a pan monocyte marker in healthy individuals. The inventors investigated whether CSF-R₁ could pull out monocytes from whole blood cells. On a fluorescent cell sorter, cells were sorted either by CD₁₄ or CSF-R₁. The CD₁₄ sort only captured two of the monocyte subsets (CD₁₄+ CD₁₆- and CD₁₄+ CD₁₆+), whereas the CSF₁R sort captured all the monocyte subsets, plus highlights a fourth, double negative population of monocytes, which is a minority in healthy individuals. The plots are representative of 3 biological replicates. This data indicates that CSF-R₁ may be a potent tool for isolating monocytes, particularly as conventional methods utilise CD₁₄ positive selection that misses monocytes, and a negative selection that consists of 7 different antibodies.

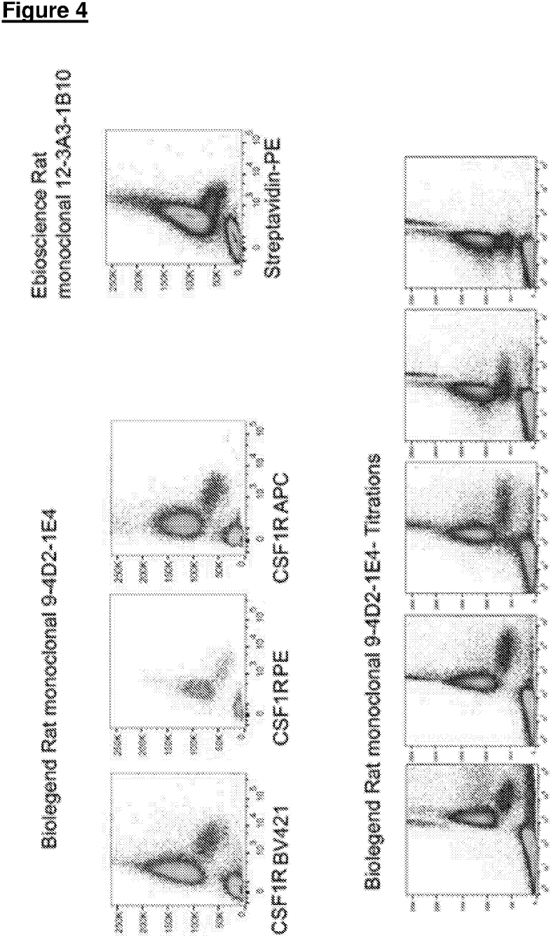

FIG. 4 shows that various commercially available CSF-R₁ antibodies can be used for CSF-R₁ monocyte detection and isolation. The inventors compared different antibodies and fluorophores for CSF-R₁. The inventors used 2 different antibody clones, the 9-₄D₂-₁E₄ rat monoclonal from Biolegend and the ₁₂-₃A₃-₁B₁₀ rat monoclonal from eBioscience. The inventors have also looked at three different fluorophores of the Biolegend antibody (BV-₄₂₁, PE and APC). There is no difference between the fluorophores, whilst the Biolegend antibody appears to be of higher affinity than the eBioscience antibody. Titrating the antibody doesn’t shift the positive CSF-R₁ population either.

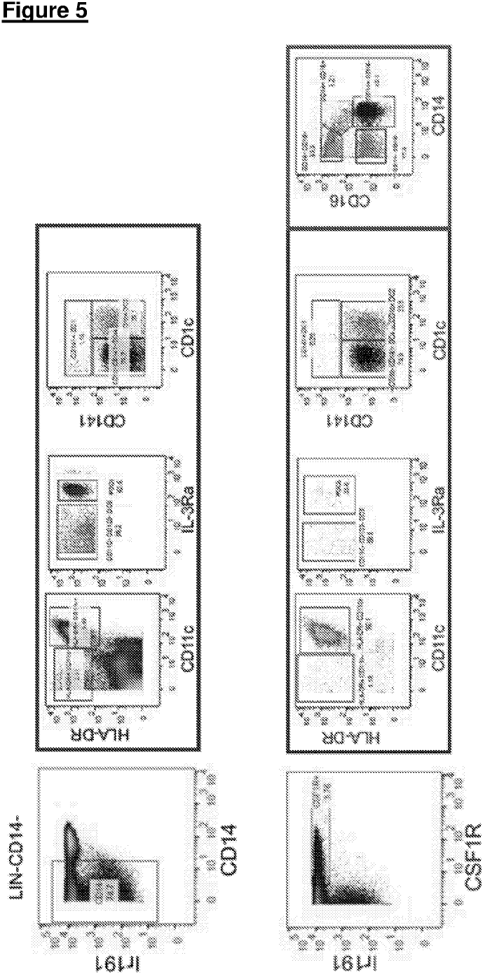

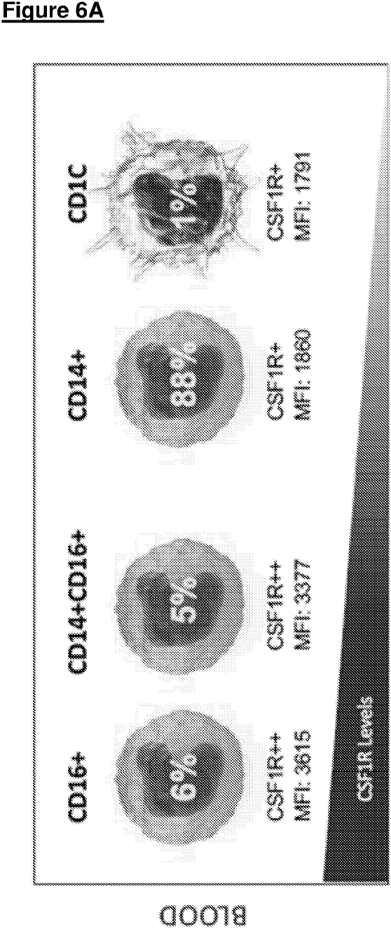

FIG. 5 shows that CSF₁R + CD14-CD16-cells are mainly CD₁C-DCs. These data was generated from CYTOF staining of PBMCs. The dendritic cells found in blood can be separated out by HLA-DR+ CD_{11c+} cells and HLA-DR+ CD_{11c-} cells. HLA-DR+ CD_{11c-}cells lead to CD₁₂₃₊ Plasmacytoid dendritic cells and a CD₁₂₃- population. The HLA-DR+ CD_{11c+} cells lead to CD₁₄₁₊ DCs, CD₁₄₁- CD_{1c+} cells and CD₁₄₁- CD_{1c-} cells. The top row displays the plots of lineage- cells (CD₃-, CD₂₀-, CD56-, CD₁₄-). The bottom row displays the CSF₁R+ cells (CD₃-, CD₂₀, CD56-). The CSF₁R cells have much less dendritic cell populations, with only a large number present in the CD₁₄₁- CD_{1c+} and CD₁₄₁- CD_{1c-} subsets.

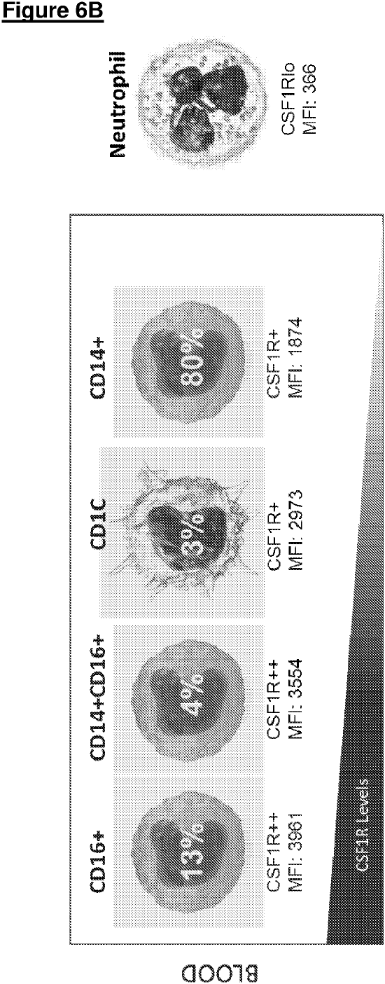

FIGS. 6A and 6B show a summary of the distribution of CSF-R₁ cells in in conventional CD₁₄/CD₁₆/CD₁C subsets.

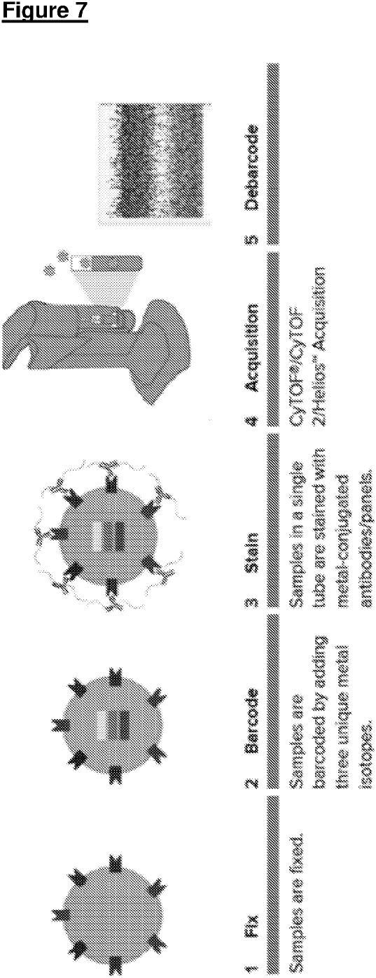

FIG. 7 shows the protocol for staining cells for CYTOF analysis.

### EXAMPLES

In this study, the inventors used a multicolour flow cytometry panel with lineage determining cytokine receptors previously described in monocytes. Although not wishing to be bound by hypothesis, the inventors believe that lineage determining cytokine receptors are directly linked with ontogeny and therefore are more informative than current monocyte markers. The inventors focused on receptors for which there was experimental evidence of expression in human monocytes and macrophages, to establish the interplay between receptors to identify a pan monocyte marker (see Table 1).

### Materials and Methods

### Blood Cells Preparation and Flow Cytometry

Peripheral venous blood was taken with informed consent from normal volunteers in accordance to ethical approval UEC/2017/052/FHMS at the University of Surrey. Blood was processed within 2 hours of venepuncture.

Leukocytes were stained in fresh whole blood in 100µl aliquots (equivalent to 1×10⁶ cells) with the appropriate antibodies and matched isotype controls. The following antibodies were used, added in a 1/100 dilution: BV₄₂₁-anti-CSF1R; BV₅₁₀-anti-CD₁₆; BV₆₅₀-anti-CD₁₄; BV₇₈₆-anti-IL3RA; FITC-anti-CSF2R; PE-anti-IL15RA; PE-Dazzle-anti-IL7R; PerCP/Cy₅.₅-anti-FLT₃; APC-anti-CSF₃R. All antibodies were purchased from Biolegend. Antibodies were incubated with the whole blood for 20mins at RT. Cells were then fixed and red blood cells lysed with RBC fix/lysis buffer (Biolegend). Cells were washed with PBS w/o Ca²⁺ and Mg²+.

Flow cytometry was performed on a triple-laser BD FACS Celesta (BD Bioscience). Cell populations were distinguished using forward light scatter (FSC-A) vs side light scatter (SSC-A), and then gated for single cells using forward light scatter height (FSC-H) vs area and live cells using the live-dead stain Zombie NIR (Biolegend). Zombie NIR staining was performed as per manufacturer’s instructions. Sorter tuning and power was kept consistent across all measurements. Panel compensation was done with single stained cells. Flow cytometry analysis was performed using the online software Cytobank™ and Flowjo™.

### Staining Cells for CYTOF Analysis

Materials:
· 3 sets of 20 barcodes in PCR tubes - 10ul each (-20° C. storage) - Included in kit
· MAXPAR Fix I buffer (5x) - 15 ml - Included in kit
· Maxpar cell staining buffer - 500 ml - Included in kit
· Maxpar 10x barcode perm buffer 50 ml - Included in kit
· Maxpar PBS - 500 ml - Included in kit
· Cell-ID Cisplatin
· Cell-ID Intercalator-Ir
   CYTOF antibody panel: Pre-fix: CSF₁R-16₃Dy; CD₁₄₁-₁₇₃Yb, CD₄₀-₁₄₂Nd, CD₃₂-₁₆₉Tm, CD₁₆-₁₄₅Nd, CD₁₀₀-₁₅₆Gd, CD₁₂₇-₁₆₅Ho
   Post-fix: CD₁₄-₁₆₀Gd, CD₆₄-₁₄₆Nd, CD₂₀₆₋₁₆₈Er, CD₁₆₃-₁₅₄Sm, CD₃₄-₁₄₈Nd, CD₈₀₋₁₆₂Dy, CD₈₆₋₁₅₀Nd, HLA-DR-₁₇₀Er, CD₃-₁₄₁-Pr, CD₁₂₃-₁₅₁Eu, CD₁₃₅-₁₅₈Gd, CXC₃R₁-₁₇₂Yb, CD₃₀₃-₁₅₃Eu, CD₁₁₇-₁₄₃Nd, CXCR₄-₁₇₅Lu, CD₂₀-₁₇₁Yb, CD_{11c-}₁₄₇Sm, CD₁₉₇-₁₅₉Tb, CD₅₆₋₁₅₅Nd, CD_{1c}-₁₄₄Nd.

Procedure for staining PBMCs:

1_{.} Collect/harvest PBMCs and re-suspend cells in MAXPAR staining buffer. Count the cells. The experiment will be performed with cell numbers of 1-3 million cells/sample.

2. Stain surface markers that can be affected by barcode fixation (i.e. CSF₁R). This may include 2-step process with secondary antibodies against the primary (i.e. anti- APC for CSF₁R). Stain in Trustain-FcX for blocking in the MAXPAR staining buffer first. Then add the primary antibodies at the desired concentration. Wash off the primary antibodies and add the secondary antibodies if required. Wash and proceed to the next step.

Antibodies added at this stage: CSF₁R; CD₁₄₁, CD₄₀, CD₃₂, CD16, CD₁₀₀, CD₁₂₇

3. Cell-ID viability staining - Prepare working solution of 10 µM Cell-ID Cisplatin (500x dilution in MAXPAR PBS); add 1 to 1 the 10uM Cisplatin to 500 µl of cells in PBS. RT incubation for 5 mins. Quench Cisplatin by washing 5-10x volume with MAXPAR cell staining buffer. Centrifuge 800 g and remove supernatant.

4. Fix and Permeabilize the cells - Re-suspend samples in 1ml of 1x Fix I buffer from the barcoding kit (Dilute 1 in 5 with MAXPAR PBS). Incubate RT 10mins. Centrifuge 1000 g 5 mins, and wash 2x with 1 ml of 1x Barcode Perm buffer (1 in 10 with MAXPAR PBS).

5. Barcode - Re-suspend each sample to be barcoded completely in 800 µl of 1x Barcode perm buffer; Re-suspend each barcode in 100 µl of 1x Barcode perm buffer and transfer them to appropriate samples. Mix immediately and incubate 30 mins RT. Gently mix halfway through. Centrifuge 1000 g 5mins, and wash 2x 2 ml MAXPAR cell staining buffer. Re-suspend in 100 µl in cell staining buffer and combine all barcoded samples in one tube (20x 100 µl = 2 ml total). Recommended to rinse sample tubes 2x 100 µl. Centrifuge 1000 g 5mins. The barcode set is composed of 6 different palladium isotopes arranged into 20 unique combinations.

6. Staining surface markers - Resuspend cells in MAXPAR staining buffer; add Fc block (Trustain FcX, 1 in 20 dilution). Add antibody panel (1 µl of antibody/3 million cells in 100 µl - so total number of cells/3 million = amount of 1µl antibody needed). For example- In barcoded sample, you would have 60 million cells in 2 ml, equivalent of 30 million in 1ml, and 3 million in 100 µl. Stain cells for 15 mins, mix, and stain for further 15 mins. Wash samples 2x with MAXPAR cell staining buffer, with centrifugation at 1000 g 5 mins.

Antibodies added at this stage: CD₁₄, CD₆₄, CD₂₀₆, CD16₃, CD₃₄, CD₈₀, CD86, HLA-DR, CD₃, CD₁₂₃, CD₁₃₅, CXC₃R₁, CD₃₀₃, CD₁₁₇, CXCR₄, CD₂₀, CD₁₁c, CD₁₉₇, CD56, CD₁c.

7. Fresh fix of cells - (Not necessary for barcoding protocol) Prepare a fresh 1.6% fix solution from 16% stock ampule. Dilute 1 in 10 the formaldehyde in MAXPAR PBS. Add 1 ml of 1.6% solution to each sample, mix and incubate 10 mins RT. Centrifuge 1000 g 5 mins.

8. Cell-ID Intercalator-Ir staining - Add 2 ml of the solution to the cells and gently mix. Solution for 1 sample is 1000x dilution of stock concentration to 125 nM working concentration, in 1 ml (1 µl of stock in 1 ml of MAXPAR FIX and PERM buffer). Incubate 1 hr RT or O/N 4C (max 48 hrs). Wash 2x MAXPAR cell staining buffer 1000 g 5 mins. Wash cells with PBS 1000 g 5 mins (Take an aliquot at this point for cell counting). Centrifuge 1000 g and leave pelleted at 4C. Take to the CYTOF.

The procedure is summarised in FIG. 7.

### Statistics

Statistical analyses were performed on GraphPad PRISM 8 program. Specific tests for each comparison are indicated in the corresponding figure legends. Graphs were plotted consistently using the mean and standard deviation error bars.

### Results

### Example 1 - Lineage Determining Cytokine Receptors in Human Monocytes And neutrophils

In order to investigate lineage determining expression in monocytes and macrophages, the inventors established a multicolour FACS panel with antibodies for CSF1R (CD115-MCSFR), CSF2RA (CD116-GMCSFR), CSF3R (CD114-GCSFR), IL15RA (CD125), IL3RA (CD123); IL7R (CD127) and FLT3 (CD135). From here on, reference to the receptors is by their accepted gene name. The receptors were selected based on experimental evidence of expression in human monocytes or macrophages (see Table 1).

TABLE 1

Summary of publications describing expression of the lineage determining cytokine receptors in human monocytes

| | | | |
|---|---|---|---|
| Cytokine receptor | Conventional physiological function | Function in human monocytes and macrophages | References in human monocytes and macrophages |
| CSF1R | Monocyte generation from haematopoietic progenitor cells, maturation to macrophages. | | (Ashmun et al., 1989; Becker et al., 1987; Boulakirba et al., 2018) |
| CSF2RA | Production, maintenance and survival of granulocytes and macrophages and dendritic cells | Production, maintenance and survival of granulocytes and macrophages; Dendritic cell generation; Promotes multinucleated giant cell formation; Inflammatory cytokine | (Agis et al., 1996; Elliot et al., 1989b; Hamilton, 2002; Mohamadzadeh et al., 2001; Sallusto and Lanzavecchia, 1994; Takahashi et al., 1997; Toyosaki-Maeda et al., 2001) |
| CSF3R | Lineage cytokine for Granulocytes, including development, activation and survival | Reduction in monocyte cytokine secretion | (Boneberg et al., 2000) |
| IL3RA | Proliferation, survival and differentiation of progenitor cells | Dendritic cell formation alongside GM-CSF; Promotes multinucleated giant cell formation | (Agis et al., 1996; Elliot et al., 1989b; Takahashi et al., 1997; Toyosaki-Maeda et al., 2001) |
| IL7R | Haematopoietic growth factor commonly associated with lymphoid cell development | In myeloid cells, stimulates dendritic cell generation alongside GM-CSF; promotes multinucleated giant cell formation; promotes IL-8 and inflammatory cytokine secretion from monocytes; generates osteoclasts | (Agis et al., 1996; Alderson et al., 1991; Chen et al., 2013; Kim et al., 2017; Standiford et al., 1992; Takahashi et al., 1997; |
| | | from monocytes; contributes to rheumatoid arthritis pathophysiology | Toyosaki-Maeda et al., 2001) |
| IL15RA | Haematopoietic growth factor commonly associated with NK cells and T cell development | Expressed on resting and activated monocytes; promotes osteoclast genesis in rheumatoid arthritis; generates cells with Langerhans morphology | (Anderson et al., 1995; Dubois et al., 2002; Mariner et al., 2002; Miranda-Carus et al., 2006; Mohamadzadeh et al., 2001) |
| FLT3 | Proliferation, survival and differentiation of progenitor cells. | Promotes dendritic cell production from monocytes; boosts dendritic cell function; boosts monocyte survival and proliferation | (Kim et al., 2015; Rappold et al., 1997) |

The inventors first evaluated surface staining of cells in whole blood. For this, they gated neutrophils, monocytes and lymphocytes according to size and granularity after red blood cell lysis (see FIGS. 1A and 1B).

Expression analysis on monocytes showed that the most abundant and selective receptor compared to neutrophils and lymphocytes is CSF1R (see FIGS. 1A-B and FIG. 5). The remaining receptors analysed did not show significant membrane expression at pan-monocyte level over matched antibody isotypes or FMO controls (see FIG. 5).

### Example 2 - CSF1R as Pan-human Monocyte Marker in CD14 and CD16 Monocyte subsets

CD14+ monocytes are the most abundant population and can mask the expression in lesser subsets. To address this, the inventors investigated the relation between lineage determining cytokine receptor and conventional monocyte subsets as defined by CD14 and CD16: CD14+CD16- monocytes, CD14+CD16+ monocytes and CD14-CD16+ monocytes (see FIGS. 2A and 2B). In a plot of CD16 vs CD14 the double negative cells have conventionally been excluded from monocytes through CD3, CD56 and CD19 expression assigning them to T cells, NK cells and B cells (Marimuthu et al., 2018; Mukherjee et al., 2015; Passlick et al., 1989). The inventors investigated the expression of the receptors in the subsets as conventionally studied.

Surface expression analysis in the monocyte subsets shows that CSF1R is highly expressed in all three subsets (see FIGS. 2A and 2B); the receptor increased with CD16 expression.

### Example 3 - CSF1R Enables Pan-monocyte Isolation by Sorting

Based on the pan-monocyte expression of CSF1R, the inventors set out to investigate its feasibility as sorting marker by comparing it with CD14. Staining was done with anti CD14, CD16 and CSF1R antibodies. CD14 sorting enables efficient monocyte purification but loss of the CD14-CD16+ population (see FIG. 3). On the other hand, CSF1R sorting captures all monocytes including CD14-CD16+ monocytes (see FIG. 3). A small population of CSF1R+ cells of ca 3% appear negative for CD14 and CD16 (see FIG. 3). The double negative population has CFS1R levels comparable to that of CD14+CD16- monocytes and express less CFS1R than CD14+CD16+ and CD14-CD16+ monocytes (see FIGS. 2A and 2B). CSF2RA was also similar between CD14-CD16-, CD14+ CD16- monocytes and CD14+CD16+ monocytes. These double negative cells appear to express similar markers to monocytes, yet are excluded by the conventional CD14 and CD16 dichotomy. FIG. 6 summarises of the distribution of CSF-R1 cells in in conventional CD14/CD16/CD1C subsets.

As shown in FIG. 4, the inventors used two different antibody clones, the 9-4D2-1E4 rat monoclonal from Biolegend and the 12-3A3-1B10 rat monoclonal from eBioscience. The inventors also looked at three different fluorophores of the Biolegend antibody (BV-421, PE and APC). There is no difference between the fluorophores, whilst the Biolegend antibody appears to be of higher affinity than the eBioscience antibody. Titrating the antibody doesn’t shift the positive CSF-R1 population either.

### Discussion

The inventors contrasted and compared simultaneously the expression of lineage determining cytokines in human monocytes versus neutrophils and within monocyte accepted subset classifications. Surprisingly, CSF1R emerged as highly expressed pan-monocyte marker. CSF1R unlike CD14 and CD16, enables the characterisation of all human monocytes in blood. Other haematopoietic receptors expressed by monocytes include IL3RA and CSF3R. Without wishing to be bound to any particular hypothesis, the inventors propose a different manner to organise monocytes in blood, which may be more informative from a cell development point of view.

The inventors’ multicolour analysis showed CSF1R receptor as a key monocyte marker. This is a receptor that is not widely accepted as human monocyte marker. There are no widespread applications for human CSF1R receptor in research, in stark contrast with the clinic where a variety of anti CSF1R tools are being trialled. The first antibody published for human CD115 was produced in the 1980s (Ashmun et al., 1989). Incidentally CD14, was published a few months later (Wright et al., 1990). CD115 original manuscript was only cited 17 times since its publication. CD14 on the contrary became the human monocyte marker of preference and is to date the main antigen used to isolate monocytes and myeloid cells from blood and fluids. CSF1R receptor has been re-described and touched upon in recent manuscripts investigating interspecies similarities and subset identity (Ingersoll et al., 2010; Schmidl et al., 2014; Wong et al., 2011).

CFS1R is an overlooked receptor and tool for monocyte quantification and isolation in humans. The inventors have now shown that CSF1R enables the identification and separation of pan-monocytes in blood. This can have repercussions in the clinic to make more accurate measurements of the monocytes and monocyte related cells in fluids. CD14 and CD16 should not be considered the only markers of monocytes and there is a bona fide CSF1R positive population that is negative for these markers, yet shares receptor profiles with the conventional monocyte subsets. This population is selective and should be considered to be part of the monocyte network.

### Conclusions

The inventors have surprisingly shown that that CSF-1R may be used as a pan-monocyte marker. The inventors were able to isolate all monocytes by FACS sorting using CSF-1R antibodies, which cannot be done with the markers CD14 or CD16 alone, and did not require any additional antibodies. The inventors have shown that CSF-1R may be used as a main monocyte marker that enables accurate isolation, quantification and a novel monocyte view from an ontogeny perspective.

### References

Agis, H., Fureder, W., Bankl, H.C., Kundi, M., Sperr, W.R., Willheim, M., BoltzNitulescu, G., Butterfield, J.H., Kishi, K., Lechner, K., and Valent, P. (1996). Comparative immunophenotypic analysis of human mast cells, blood basophils and monocytes. Immunology 87,535-543.

Alderson, M.R., Tough, T.W., Ziegler, S.F., and Grabstein, K.H. (1991). Interleukin-7 induces cytokine secretion and tumoricidal activity by human peripheral-blood monocytes. J. Exp. Med. 173, 923-930.

Anderson, D.M., Kumaki, S., Ahdieh, M., Bertles, J., Tometsko, M., Loomis, A., Giri, J., Copeland, N.G., Gilbert, D.J., Jenkins, N.A., et al. (1995). Functional-characterization of the human interleukin-15 receptor-alpha chain and close linkage of il15ra and il2ra genes. J. Biol. Chem. 270, 29862-29869.

Ashmun, R.A., Look, A.T., Roberts, W.M., Roussel, M.F., Seremetis, S., Ohtsuka, M., and Sherr, C.J. (1989). Monoclonal-antibodies to the human csf-1 receptor (c-fms proto-oncogene product) detect epitopes on normal mononuclear phagocytes and on human myeloid leukemic blast cells. Blood 73, 827-837.

Becher, B., Tugues, S., and Greter, M. (2016). GM-CSF: From Growth Factor to Central Mediator of Tissue Inflammation. Immunity 45, 963-973.

Becker, S., Warren, M.K., and Haskill, S. (1987). Colony-stimulating factor-induced monocyte survival and differentiation into macrophages in serum-free cultures. J. Immunol. 139, 3703-3709.

Boneberg, E.M., Hareng, L., Gantner, F., Wendel, A., and Hartung, T. (2000). Human monocytes express functional receptors for granulocyte colony-stimulating factor that mediate suppression of monokines and interferon-gamma. Blood 95, 270-276.

Borriello, F., Iannone, R., Di Somma, S., Loffredo, S., Scamardella, E., Galdiero, M.R., Varricchi, G., Granata, F., Portella, G., and Marone, G. (2016). GM-CSF and IL-3 Modulate Human Monocyte TNF-alpha Production and Renewal in In Vitro Models of Trained Immunity. Front Immunol 7, 680.

Boulakirba, S., Pfeifer, A., Mhaidly, R., Obba, S., Goulard, M., Schmitt, T., Chaintreuil, P., Calleja, A., Furstoss, N., Orange, F., et al. (2018). IL-34 and CSF-1 display an equivalent macrophage differentiation ability but a different polarization potential. Sci Rep 8, 11.

Brugger, W., Kreutz, M., and Andreesen, R. (1991). Macrophage colony-stimulating factor is required for human monocyte survival and acts as a cofactor for their terminal differentiation to macrophages invitro. J. Leukoc. Biol. 49, 483-488.

Chen, Z., Kim, S.J., Chamberlain, N.D., Pickens, S.R., Volin, M.V., Volkov, S., Arami, S., Christman, J.W., Prabhakar, B.S., Swedler, W., et al. (2013). The Novel Role of IL-7 Ligation to IL-7 Receptor in Myeloid Cells of Rheumatoid Arthritis and Collagen-Induced Arthritis. J. Immunol. 190, 5256-5266.

Dubois, S., Mariner, J., Waldmann, T.A., and Tagaya, Y. (2002). IL-15R alpha recycles and presents IL-15 in trans to neighboring cells. Immunity 17, 537-547.

Elliott, M.J., Vadas, M.A., Eglinton, J.M., Park, L.S., To, L.B., Cleland, L.G., Clark, S.C., and Lopez, A.F. (1989a). Recombinant human interleukin-3 and granulocyte-macrophage colony-stimulating factor show common biological effects and binding characteristics on human monocytes. Blood 74, 2349-2359.

Elliott, M.J., Vadas, M.A., Eglinton, J.M., Park, L.S., To, L.B., Cleland, L.G., Clark, S.C., and Lopez, A.F. (1989b). Recombinant human interleukin-3 and granulocyte-macrophage colony-stimulating factor show common biological effects and binding characteristics on human-monocytes. Blood 74, 2349-2359.

Eyles, J.L., Hickey, M.J., Norman, M.U., Croker, B.A., Roberts, A.W., Drake, S.F., James, W.G., Metcalf, D., Campbell, I.K., and Wicks, I.P. (2008). A key role for G-CSF-induced neutrophil production and trafficking during inflammatory arthritis. Blood 112, 5193-5201.

Goyert, S.M., Ferrero, E., Rettig, W.J., Yenamandra, A.K., Obata, F., and Lc Beau, M.M. (1988). The CD14 monocyte differentiation antigen maps to a region encoding growth factors and receptors. Science 239, 497-500.

Hamilton, J.A. (2002). GM-CSF in inflammation and autoimmunity. Trends Immunol. 23, 403-408.

Hansen, G., Hercus, T.R., McClure, B.J., Stomski, F.C., Dottore, M., Powell, J., Ramshaw, H., Woodcock, J.M., Xu, Y.B., Guthridge, M., et al. (2008). The structure of the GM-CSF receptor complex reveals a distinct mode of cytokine receptor activation. Cell 134, 496-507.

Ingersoll, M.A., Spanbroek, R., Lottaz, C., Gautier, E.L., Frankenberger, M., Hoffmann, R., Lang, R., Haniffa, M., Collin, M., Tacke, F., et al. (2010). Comparison of gene expression profiles between human and mouse monocyte subsets. Blood 115, e10-19.

Karlsson, K.R., Cowley, S., Martinez, F.O., Shaw, M., Minger, S.L., and James, W. (2008). Homogeneous monocytes and macrophages from human embryonic stem cells following coculture-free differentiation in M-CSF and IL-3. Exp Hematol 36, 1167-1175.

Kim, J.H., Sim, J.H., Lee, S., Seol, M.A., Ye, S.K., Shin, H.M., Lee, E.B., Lee, Y.J., Choi, Y.J., Yoo, W.H., et al. (2017). Interleukin-7 Induces Osteoclast Formation via STAT5, Independent of Receptor Activator of NF-kappaB Ligand. Front. Immunol. 8, 13.

Kim, S.W., Choi, S.M., Choo, Y.S., Kim, I.K., Song, B.W., and Kim, H.S. (2015). Flt3 Ligand Induces Monocyte Proliferation and Enhances the Function of Monocyte-Derived Dendritic Cells In Vitro. J. Cell. Physiol. 230, 1740-1749.

Kitamura, T., Sato, N., Arai, K., and Miyajima, A. (1991). Expression cloning of the human IL-3 receptor cdna reveals a shared beta-subunit for the human IL-3 and GM-CSF receptors. Cell 66, 1165-1174.

Mahaweni, N.M., Olieslagers, T.I., Rivas, 1.0., Molenbroeck, S.J.J., Groeneweg, M., Bos, G.M.J., Tilanus, M.G.J., Voorter, C.E.M., and Wieten, L. (2018). A comprehensive overview of FCGR3A gene variability by full-length gene sequencing including the identification of V158F polymorphism. Sci Rep 8, 15983.

Marimuthu, R., Francis, H., Dervish, S., Li, S.C.H., Medbury, H., and Williams, H. (2018). Characterization of Human Monocyte Subsets by Whole Blood Flow Cytometry Analysis. J Vis Exp.

Mariner, J.M., Mamane, Y., Hiscott, J., Waldmann, T.A., and Azimi, N. (2002). IFN regulatory factor 4 participates in the human T cell lymphotropic virus type I-mediated activation of the IL-15 receptor alpha promoter. J. Immunol. 168, 5667-5674.

Martinez, F.O. (2009). The transcriptome of human monocyte subsets begins to emerge. J Biol 8, 99.

Miranda-Carus, M.E., Benito-Miguel, M., Balsa, A., Cobo-Ibanez, T., de Ayala, C.P., Pascual-Salcedo, D., and Martin-Mola, E. (2006). Peripheral blood T lymphocytes from patients with early rheumatoid arthritis express RANKL and interleukin-15 on the cell surface and promote osteoclastogenesis in autologous monocytes. Arthritis Rheum. 54, 1151-1164.

Mohamadzadeh, M., Berard, F., Essert, G., Chalouni, C., Pulendran, B., Davoust, J., Bridges, G., Palucka, A.K., and Banchereau, J. (2001). Interleukin 15 skews monocyte differentiation into dendritic cells with features of Langerhans cells. J. Exp. Med. 194, 1013-1019.

Mukherjee, R., Kanti Barman, P., Kumar Thatoi, P., Tripathy, R., Kumar Das, B., and Ravindran, B. (2015). Non-Classical monocytes display inflammatory features: Validation in Sepsis and Systemic Lupus Erythematous. Sci Rep 5, 13886.

Passlick, B., Flieger, D., and Ziegler-Heitbrock, H.W. (1989). Identification and characterization of a novel monocyte subpopulation in human peripheral blood. Blood 74, 2527-2534.

Pickens, S.R., Chamberlain, N.D., Volin, M.V., Pope, R.M., Talarico, N.E., Mandelin, A.M., and Shahrara, S. (2011). Characterization of Interleukin-7 and Interleukin-7 Receptor in the Pathogenesis of Rheumatoid Arthritis. Arthritis Rheum. 63, 2884-2893.

Rappold, I., Ziegler, B.L., Kohler, I., Marchetto, S., Rosnet, O., Birnbaum, D., Simmons, P.J., Zannettino, A.C.W., Hill, B., Neu, S., et al. (1997). Functional and phenotypic characterization of cord blood and bone marrow subsets expressing FLT3 (CD135) receptor tyrosine kinase. Blood 90, 111-125.

Sallusto, F., and Lanzavecchia, A. (1994). Efficient presentation of soluble-antigen by cultured human dendritic cells is maintained by granulocyte-macrophage colony-stimulating factor plus interleukin-4 and down-regulated by tumor-necrosis-factor-alpha. J. Exp. Med. 179, 1109-1118.

Schmidl, C., Renner, K., Peter, K., Eder, R., Lassmann, T., Balwierz, P.J., Itoh, M., Nagao-Sato, S., Kawaji, H., Carninci, P., et al. (2014). Transcription and enhancer profiling in human monocyte subsets. Blood 123, e90-99.

Sonderegger, I., Iezzi, G., Maier, R., Schmitz, N., Kurrer, M., and Kopf, M. (2008). GM-CSF mediates autoimmunity by enhancing IL-6-dependent Th17 cell development and survival. J. Exp. Med. 205, 2281-2294.

Standiford, T.J., Strieter, R.M., Allen, R.M., Burdick, M.D., and Kunkel, S.L. (1992). Il-7 up-regulates the expression of IL-8 from resting and stimulated human blood monocytes. J. Immunol. 149, 2035-2039.

Takahashi, K., Honeyman, M.C., and Harrison, L.C. (1997). Dendritic cells generated from human blood in granulocyte macrophage-colony stimulating factor and interleukin-7. Hum. Immunol. 55, 103-116.

Toyosaki-Maeda, T., Takano, H., Tomita, T., Tsuruta, Y., Maeda-Tanimura, M., Shimaoka, Y., Takahashi, T., Itoh, T., Suzuki, R., and Ochi, T. (2001). Differentiation of monocytes into multinucleated giant bone-resorbing cells: two-step differentiation induced by nurse-like cells and cytokines. Arthritis Res. 3, 306-310.

Villani, A.C., Satija, R., Reynolds, G., Sarkizova, S., Shekhar, K., Fletcher, J., Griesbeck, M., Butler, A., Zheng, S., Lazo, S., et al. (2017). Single-cell RNA-seq reveals new types of human blood dendritic cells, monocytes, and progenitors. Science 356.

Wang, Y.M., Bugatti, M., Ulland, T.K., Vermi, W., Gilfillan, S., and Colonna, M. (2016). Nonredundant roles of keratinocyte-derived IL-34 and neutrophil-derived CSF1 in Langerhans cell renewal in the steady state and during inflammation. Eur. J. Immunol. 46, 552-559.

Wang, Y.M., Szretter, K.J., Vermi, W., Gilfillan, S., Rossini, C., Cella, M., Barrow, A.D., Diamond, M.S., and Colonna, M. (2012). IL-34 is a tissue-restricted ligand of CSF1R required for the development of Langerhans cells and microglia. Nat. Immunol. 13, 753-+.

Wong, K.L., Tai, J.J., Wong, W.C., Han, H., Sem, X., Yeap, W.H., Kourilsky, P., and Wong, S.C. (2011). Gene expression profiling reveals the defining features of the classical, intermediate, and nonclassical human monocyte subsets. Blood 118, e16-31.

Wright, S.D., Ramos, R.A., Tobias, P.S., Ulevitch, R.J., and Mathison, J.C. (1990). CD14, a receptor for complexes of lipopolysaccharide (LPS) and LPS binding protein. Science 249, 1431-1433.

1. The use of an antibody, or antigen-binding fragment thereof, which binds to colony-stimulating factor 1 receptor (CSF-1R), or a variant or fragment thereof, to isolate a monocyte and/or a monocyte-derived cell from a biological sample.

2. The use of the antibody, or antigen-binding fragment thereof, according to claim 1, wherein the biological sample is a human or murine biological sample.

3. The use of the antibody, or antigen-binding fragment thereof, according to claim 1, wherein the biological sample is a tissue or a biological fluid.

4. The use of the antibody, or antigen-binding fragment thereof, according to claim 1, wherein the biological sample is blood.

5. The use according to claim 1, wherein the monocyte derived cell is a macrophage or a myeloid lineage dendritic cell.

6. The use according to claim 1, wherein isolation comprise use of florescence-activated cell sorting (FACS), magnetic activated cell separation or buoyancy activated cell separation.

7. The use according to claim 6, wherein the magnetic activated cell separation is immunomagnetic cell separation or affinity magnetic cell separation.

8. The use of the antibody, or antigen-binding fragment thereof, according to claim 1, wherein CSF-R1 comprises or consists of an amino acid sequence as substantially as set out in SEQ ID No: 1, or a variant or fragment thereof.

9. The use of the antibody, or antigen-binding fragment thereof, according to claim 1, wherein the antibody, or antigen binding fragment thereof is used to isolate:
i) a CD14 positive and CD16 positive monocyte;
ii) a CD14 negative and CD16 positive monocyte;
iii) a CD14 positive and CD16 negative monocyte; and/or
iv) a CD14 negative and CD16 negative monocyte-derived myeloid lineage dendritic cell.

10. The use of the antibody, or antigen-binding fragment thereof, according to claim 1, wherein the antibody, or antigen binding fragment thereof is used to isolate:
i) a CD14 positive and CD16 positive monocyte;
ii) a CD14 negative and CD16 positive monocyte;
iii) a CD14 positive and CD16 negative monocyte; and
iv) a CD14 negative and CD16 negative monocyte-derived myeloid lineage dendritic cell.

11. A method of isolating a monocyte and/or a monocyte-derived cell from a biological sample, the method comprising:
i) contacting a biological sample comprising a monocyte and/or a monocyte-derived cell with the antibody, or antigen-binding fragment thereof, according to claim 1; and
ii) collecting a monocyte and/or a monocyte-derived cell present in the biological sample that binds to the antibody or antigen-binding fragment thereof, thereby isolating the monocyte and/or a monocyte-derived cell.

12. The method according to claim 11, wherein the method is used to isolate:
i) a CD14 positive and CD16 positive monocyte;
ii) a CD14 negative and CD16 positive monocyte;
iii) a CD14 positive and CD16 negative monocyte; and/or
iv) a CD14 negative and CD16 negative monocyte-derived myeloid lineage dendritic cell.

13. The method according to claim 11, wherein the method is used to isolate:
i) a CD14 positive and CD16 positive monocyte;
ii) a CD14 negative and CD16 positive monocyte;
iii) a CD14 positive and CD16 negative monocyte; and
iv) a CD14 negative and CD16 negative monocyte-derived myeloid lineage dendritic cell.

14. The method according to claim 11, wherein the method comprises use of florescence-activated cell sorting (FACS), magnetic activated cell separation or buoyancy activated cell separation.

15. The method according to claim 14, wherein the magnetic activated cell separation is immunomagnetic cell separation or affinity magnetic cell separation.

16. The method according to claim 11, wherein the method comprises use of FACS, wherein the method comprises:
i) contacting a biological sample that comprises a monocyte and/or a monocyte-derived cell with a fluorescently labelled antibody, or antigen-binding fragment thereof, according to claim 1; and
ii) sorting cells present in the biological sample based on their fluorescence; and
iii) collecting a monocyte and/or a monocyte-derived cell present in the biological sample that are bound to the fluorescently labelled antibody, thereby isolating a monocyte and/or a monocyte-derived cell that is present in the biological sample.

17. The method according to claim 11, wherein the biological sample is a tissue or a biological fluid.

18. A method of detecting a monocyte and/or a monocyte-derived cell present in a biological sample, the method comprising:
i) contacting a biological sample comprising a monocyte and/or a monocyte-derived cell with the antibody, or antigen-binding fragment thereof, according to claim 1; and
ii) detecting a monocyte and/or a monocyte-derived cell present in the biological sample by a detection means.

19. The method according to claim 18, wherein the detection means comprises immunostaining or FACS.


SEQUENCE LISTING (11 sequences)

SEQ ID NO: 1 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 2 - protein, 112 aa, Homo sapiens
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTV

SEQ ID NO: 3 - protein, 8 aa, Homo sapiens
DQRLYFDV

SEQ ID NO: 4 - protein, 16 aa, Homo sapiens
VIWTDGGTNYAQKLQG

SEQ ID NO: 5 - protein, 5 aa, Homo sapiens
SYDIS

SEQ ID NO: 6 - protein, 106 aa, Homo sapiens
DIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIK

SEQ ID NO: 7 - protein, 8 aa, Homo sapiens
QQSFSYPT

SEQ ID NO: 8 - protein, 7 aa, Homo sapiens
AASNRYT

SEQ ID NO: 9 - protein, 11 aa, Homo sapiens
RASEDVNTYVS

SEQ ID NO: 10 - protein, 446 aa, Homo sapiens
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 213 aa, Homo sapiens
DIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC